(19) United States
(12) Patent Application Publication
(10) Pub. No.: US 2021/0055249 A1
(43) Pub. Date: Feb. 25, 2021

(54) ELECTRODE MATERIAL FOR SENSOR, ELECTRODE FOR SENSOR, SENSOR, AND BIOSENSOR

(71) Applicant: Nitto Denko Corporation, Ibaraki-shi, Osaka (JP)

(72) Inventors: Takashi ODA, Ibaraki-shi, Osaka (JP); Shotaro MASUDA, Ibaraki-shi, Osaka (JP)

(21) Appl. No.: 16/967,258

(22) PCT Filed: Jan. 21, 2019
(86) PCT No.: PCT/JP2019/001700

§ 371 Date: Aug. 4, 2020

(30) Foreign Application Priority Data
Feb. 8, 2018 (JP) 2018-020761

(51) Int. Cl. G01N 27/30; C01B 32/158; G01N 33/543; C12Q 1/00; G01N 27/327

(52) U.S. Cl. CPC G01N 27/308; C01B 32/158; G01N 33/5438; C12Q 1/001; B82Y 15/00; C01B 2202/06; C01B 2202/22; C01P 2004/03; G01N 27/3271

(57) ABSTRACT

The present invention provides an electrode material for a sensor, the material includes a sheet-like carbon nanotube assembly including a plurality of carbon nanotubes, wherein a length of each carbon nanotube extends from one surface of the carbon nanotube assembly toward the other surface thereof, and the carbon nanotube assembly includes a low orientation portion of the carbon nanotubes.

### TECHNICAL FIELD

The present invention relates to an electrode material for a sensor, an electrode for a sensor, a sensor, and a biosensor.

### BACKGROUND OF THE INVENTION

Carbon nanotubes are tube-shaped material with a diameter of 1 nm to several tens of nm obtained by rolling a graphene sheet (a layer made of a 6-membered carbon ring) into a cylindrical shape. Carbon nanotubes are excellent conductivity, chemical stability, and heat conductivity. Therefore, carbon nanotubes have been attracting attention as electrode materials for sensors such as chemical sensors and biosensors. For example, Patent Document 1 describes a sensor including an electrode formed by directly forming carbon nanotubes on a metal surface.

### RELATED-ART DOCUMENT

### Patent Documents

Patent document 1: Japanese Unexamined Patent Publication No. 2008-64724

### SUMMARY OF THE INVENTION

### Problems to be Solved by the Invention

However, in the electrode material described in Patent Document 1, there is room for improvement in enhancing the detection sensitivity of the sensor.

In view of the above, an object of one aspect of the present invention provides an electrode material for a sensor capable of producing a highly sensitive sensor.

### Means for Solving the Problems

One aspect of the present invention is to provide an electrode material for a sensor, the material includes a sheet-like carbon nanotube assembly including a plurality of carbon nanotubes, wherein a length of each carbon nanotube extends from one surface of the carbon nanotube assembly toward the other surface thereof, and the carbon nanotube assembly includes a low orientation portion in the carbon nanotubes.

### Effect of the Invention

According to one aspect of the present invention, the present invention is able to provide an electrode material for a sensor capable of producing a highly sensitive sensor.

### BRIEF DESCRIPTION OF THE DRAWINGS

FIG. 1 shows the schematic diagram of the carbon nanotube assembly in one aspect of the present invention.

FIG. 2 shows the schematic diagram of the production apparatus of the carbon nanotube assembly in one aspect of the present invention.

FIG. 3 shows the schematic diagram which shows the surface of the electrode material in the biosensor in one aspect of the invention.

FIG. 4 shows the SEM photograph which shows the cross-section in the thickness direction (a) at intermediate part and (b) in the vicinity of the main part of the carbon nanotube assembly in one aspect of the present invention.

FIG. 5 shows the SEM photograph which shows the cross-section in the thickness direction (a) at intermediate part and (b) in the thickness direction in the vicinity of the main part of the carbon nanotube assembly in the conventional invention.

FIG. 6 shows the schematic diagram of the sensor used in the present Examples.

### DETAILED DESCRIPTION

One aspect of the present invention provides an electrode material for a sensor, the material includes a sheet-like carbon nanotube assembly including a plurality of carbon nanotubes (sometimes referred to as CNT), wherein a length of each carbon nanotube extends from one surface of the carbon nanotube assembly toward the other surface thereof, and the carbon nanotube assembly includes a low orientation portion in the carbon nanotubes.

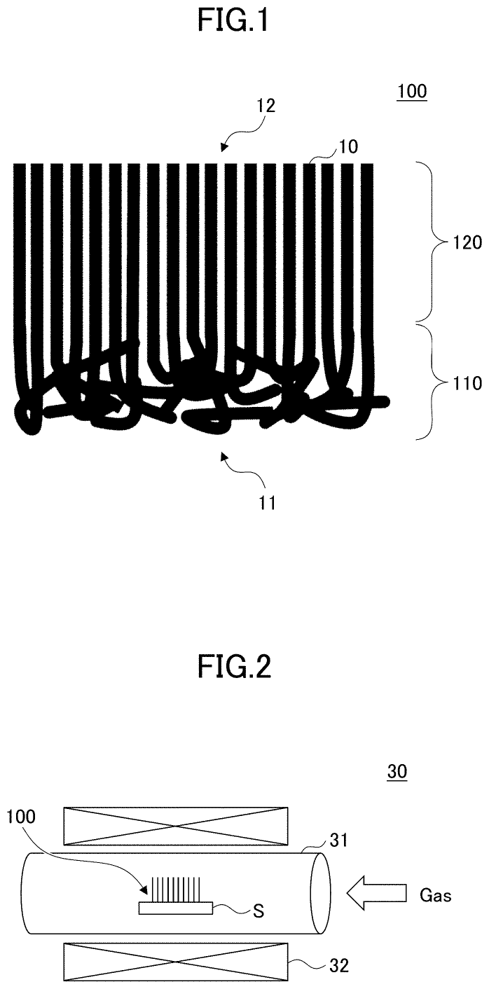

FIG. 1 schematically shows an assembly 100 of carbon nanotubes according to this embodiment. As illustrated, the carbon nanotube assembly 100 includes a plurality of carbon nanotubes 10. The carbon nanotubes have a structure in which the length of each carbon nanotubes is oriented from one main surface (lower surface) 11 of the sheet-like carbon nanotube assembly to the other main surface (upper surface) 12. That is, each of the carbon nanotubes 10 as a whole extends in a direction substantially perpendicular to the main surfaces 11 and 12 of the carbon nanotube assembly (sheet) 100. Such an extended state indicates that one end of each carbon nanotubes 10 reaches one main surface 11 of the sheet and the other end reaches the other main surface 12.

This sheet-like carbon nanotube assembly has a low orientation portion in at least one region of both main surfaces of the sheet. In the Figures, the carbon nanotube assembly 100 has a low orientation portion 110 in the vicinity of one surface 11 (near the surface 11) of the carbon nanotube assembly 100, and the carbon nanotube assembly has a high orientation portion 120 on the opposite side of the low orientation portion 110 (on the side of the other surface 12 of the carbon nanotube assembly 100). When the carbon nanotube assembly 100 is used as an electrode material, one surface 11 is adhered to an appropriate electrode substrate such as metals, and the other surface 12 serves as a surface for detecting a target.

As illustrated in FIG. 1, each of the carbon nanotubes in the low orientation portion 110 is meandering in the middle, and there are many portions that do not extend vertically when viewed microscopically. That is, each carbon nanotubes have the portion which the orientation angle is not in a direction substantially perpendicular to the main surface. For example, each carbon nanotube has a portion that deviates from the angles ranges such as 85 to 95°, 80 to 100°, 75 to 105°, or 70 to 110° with respect to the surface of the sheet. In addition, the low orientation portion 110 may extend in various directions of the plurality of carbon nanotubes when the carbon nanotubes are microscopically observed. Therefore, the low orientation portion is low in orientation degree. Such a plurality of carbon nanotubes 10 are entangled with each other to form a relatively dense mesh-like structure (mesh structure) in the low orientation portion 110.

In the present embodiment, because the carbon nanotube assembly has the low orientation portion as described above, the adjacent or neighboring carbon nanotubes can contact each other at a plurality of locations, and the carbon nanotubes can be electrically connected at the plurality of locations. In other words, the low orientation portion can form a conductive fibrous network structure that is three-dimensionally intertwined. Thereby, the number of conductive paths in the carbon nanotube assembly can be increased, and the number of carbon nanotubes that sense the target to be detected can be increased (i.e., the density of electrodes that react with the target to be detected can be increased). More specifically, even if there is only one carbon nanotube in contact with the object to be detected, the electric current will flow through the plurality of carbon nanotubes due to the above-mentioned mesh structure. As a result, an electric current can be acquired from a plurality of carbon nanotubes. Therefore, even a target to be detected in low concentration can be reliably detected.

In addition, even if a part of a plurality of carbon nanotubes or a part of one carbon nanotube is partially damaged due to aging or the like, a target still can be detected because the carbon nanotube assembly is capable of securing the path by providing a plurality of current paths.

Furthermore, when comparing among carbon nanotube assemblies containing the same number and the same diameter of carbon nanotubes, the length of each carbon nanotube can be increased in the carbon nanotube assembly having the low orientation portion. In addition, the density of the carbon nanotubes contained in the assembly also increases. As a result, the surface area (specific surface area) of the carbon nanotubes per volume of the carbon nanotube assembly (sheet) can be increased. When the carbon nanotube assembly is used as a sensor, the surface of the carbon nanotube may be modified by a substance (a substance that becomes a reaction site) that can bind to a target to be detected or attract a target to be detected depending on the type of the target to be detected. Therefore, according to this embodiment, the area of the carbon nanotube surface to be modified can be increased. Thereby, an amount of substance to be able to carry can be increased, and as a result, the detection sensitivity can be increased.

The detection sensitivity of the electrode can be evaluated based on, for example, the magnitude of the electrochemical signal (electric current value at the reduction peak or the oxidation peak, etc.) measured by the CV method (cyclic voltammetry) for the electrolyte solution.

In addition, in the present embodiment, the physical connection between the carbon nanotubes in the plane direction can be enhanced due to the presence of the low orientation portion in the carbon nanotube assembly. Therefore, the mechanical strength of the carbon nanotube assembly can be improved.

Furthermore, the carbon nanotube assembly can be formed into an independent sheet-like carbon nanotube assembly without having a support of another substrate or the like. That is, the carbon nanotube assembly obtained by growing the carbon nanotubes on the substrate can be easily separated from the substrate using, for example, tweezers or the like, and transferred to another surface. Therefore, when the carbon nanotube assembly according to the present embodiment is used for a sensor, the grown carbon nanotube assembly can be transferred to an appropriate surface to form a desired sensor. In other words, the use of the carbon nanotube assembly of the present embodiment expands the choice of electrode substrates.

More specifically, the low orientation portion of carbon nanotube may be a portion where the carbon nanotube has a distribution of orientation angles and a portion having a predetermined orientation degree. In the present specification, the orientation degree of the carbon nanotubes is a proportion of the total length of the portions of the carbon nanotubes having orientation angles of 70 to 110° with respect to the one surface or the other surface based on 100% of the total length of the carbon nanotubes. In order to obtain the orientation degree, for example, a cross-sectional image of the carbon nanotube assembly (sheet) cut in a direction perpendicular to the main surface of the sheet (cut along the thickness direction of the sheet) is obtained by a scanning electron microscope (SEM) or a transmission electron microscope (TEM), and then the image is analyzed.

In the image analysis, all carbon nanotubes in the image can be extracted as needle shape particles, and the orientation angle of each extracted needle shape particle can be obtained. When the orientation angle of each needle shape particle varies, the needle shape particle is divided per the angle variation occurs at a predetermined angle, for example, the orientation angle with respect to the main surface of the sheet is different by 10°. Then, the length of each of the divided portions is obtained. Then, in the image, the proportion of the total length of the portions of the carbon nanotubes having orientation angles of 70 to 110° with respect to the main surface of the carbon nanotube assembly based on the total length of the all carbon nanotubes is determined. That is, (the total length of the portions of the carbon nanotubes having orientation angles of 70 to 110°)/(the total length of carbon nanotubes)×100 is performed from the acquired image. For such an analysis, image analysis software such as Winroof (manufactured by Mitani Corporation), which exhibits a function individually separates needle shape particle, can be used.

In the low orientation portion, the orientation degree of carbon nanotubes is 75% or less, preferably 65% or less, and more preferably 50% or less. By adjusting the orientation degree of the carbon nanotubes to the above range, the above-mentioned mesh structure can be denser. Accordingly, the effects such that enhancing the detection sensitivity by increasing the number of conductive paths, increasing the specific surface area, and forming a sheet-like carbon nanotube assembly exerted by the above can be more enhanced.

In the form of FIG. 1, the low orientation portion 110 is formed in the vicinity of the one surface 11 of the carbon nanotube assembly 100, and the other portion of the carbon nanotube assembly 100 is the high orientation portion 120. However, the low orientation portion 110 may be formed in the vicinity of the other surface 12 of the carbon nanotube assembly 100. Further, the low orientation portion may be formed in the vicinity of both of the one surface 11 and the other surface 12, or may be formed from one surface 11 or the other surface 12 to the central portion of the carbon nanotube assembly 100. Also, the low orientation portion 110 may be formed in the portion other than the one surface 12 or the other surface 12 of the carbon nanotube assembly 100. Further, the entire carbon nanotube assembly 100 may be the low orientation portion 110.

The low orientation portion 110 is preferably formed at least in a region in the vicinity of the one surface 11 of the carbon nanotube assembly 100. In this case, the area in the vicinity of the one surface 11 can be an area up to 20 μm in the thickness direction from the one surface 11. That is, the low orientation portion 110 is preferably formed in the area up to 20 μm from the one surface 11 (a portion from one surface 11 to a portion at 20 μm in the thickness direction), and the low orientation portion 110 is preferably the entire portion up to the predetermined thickness. The predetermined thickness up to 20 μm may be preferably 15 μm, more preferably 10 μm, further preferably 4 μm, and further more preferably 2 μm.

The low orientation portion may be a portion in which the average value of the number of places where the carbon nanotube contacts the carbon nanotube itself or another carbon nanotube is 3 or more per the length 1 μm of the carbon tube.

In addition, in the carbon nanotube assembly, the orientation angles of the carbon nanotubes have a distribution, that is, the carbon nanotube assembly preferably have a variation. Here, the variation in the orientation angle in the low orientation portion in the carbon nanotube assembly may be greater than that of the variation in the high orientation portion in the carbon nanotube assembly (for example, the values of the standard deviation, the dispersion, etc. are larger). Due to the large variation in the orientation angle of the carbon nanotubes, a more apparent mesh structure in which the carbon nanotubes are entangled with each other can be obtained.

The thickness of the low orientation portion in the carbon nanotube assembly (the total thickness when a plurality of low orientation portion in carbon nanotube assembly exists when viewed in the thickness direction) is preferably 0.001% to 80%, more preferably 0.01% to 50%, further more preferably 0.05% to 30%, and particularly preferably 0.1% to 20% with respect to the total thickness of the carbon nanotube assembly (the sum of the thickness of the high orientation portion and the low orientation portion). By adjusting the thickness of the low orientation portion to 0.001% or more of the entire sheet, the conductive path and surface area can be increased, the sensitivity can be further improved, and resulting in firmly maintain the sheet-like form of the carbon nanotube assembly. Further, in the form in which the thickness of the low orientation portion is 50% or less of the entire sheet, it is preferable in that increasing the ratio of high orientation portion where the target to be detected easily enters between carbon nanotubes, when the target to be detected is detected by capturing the relatively large size between the carbon nanotubes.

The total thickness of the carbon nanotube assembly is, for example, 10 to 5000 μm, preferably 50 to 4000 μm, more preferably 100 to 3000 μm, and further preferably 300 to 2000 μm. The thickness of the carbon nanotube assembly may be, for example, an average value of three randomly extracted points within 0.2 mm or more from the end in the plane direction of the carbon nanotube assembly layer.

The high orientation portion in the carbon nanotube assembly is a portion having relatively high orientation and having an orientation degree of a predetermined value or more. In the form of FIG. 1, the high orientation portion 120 is provided on the side of the other surface 12. When the other surface 12 is exposed to a target to be detected (when the one surface 11 is a surface to be adhered to an electrode substrate), the side of the high orientation portion 120 can easily contact with the target to be detected. Therefore, when the target to be detected is captured between the carbon nanotubes for detection, even if a target to be detected is relatively large, the target to be detected can easily enter between the carbon nanotubes. From this standpoint, the high orientation portion 120 is preferably formed on the side of the other surface 12 of the carbon nanotube assembly 100.

The high orientation portion of the carbon nanotube assembly can be a portion where the orientation degree of carbon nanotubes exceeds 75%. Therefore, the high orientation portion may include carbon nanotubes in which orientation angle is a direction (direction other than 90°) deviating from a direction perpendicular to the surface of the sheet. Here, the orientation degree of the carbon nanotubes in the high orientation portion is 90% or less, preferably 85% or less, more preferably less than 84%, and further more preferably 80% or less from the viewpoint of improving the sensitivity by increasing the contact between carbon nanotubes even in the high orientation portion in carbon nanotube and of increasing the strength of the carbon nanotube assembly. On the other hand, when a target to be detected is captured between the carbon nanotubes for detection, even if a target to be detected is relatively large, the target to be detected can easily enter between the carbon nanotubes. The orientation degree can be 80% or more, preferably 85% or more, and more preferably 90% or more.

The carbon nanotubes included in the carbon nanotube assembly in the present embodiment may be a single-walled carbon nanotube (SWCNT) or a multi-walled carbon nanotube (MWCNT). When a multi-walled carbon nanotube is used, the electrode material according to the present embodiment provided with carbon nanotube assembly behaves like a metal in that it exhibits excellent conductivity.

In the case of a multi-wall carbon nanotube, the distribution width of the number distribution of carbon nanotube walls (difference between the maximum value and the minimum value of the number of carbon nanotube walls) is 30 walls or less, preferably 20 walls or less, and more preferably 15 walls or less.

The average number of carbon nanotube walls is preferably 2 to 30 walls and more preferably 3 to 15 walls. The maximum number of walls of carbon nanotubes is preferably 40 walls or less and more preferably 30 walls or less. The minimum number of carbon nanotube walls is preferably 20 walls or less and more preferably 10 walls or less.

Further, the relative frequency of the mode of the number distribution of the walls of the carbon nanotubes is preferably 40% or less. The mode of the number distribution of the walls of the carbon nanotubes is preferably shown in 1 to 30 walls and more preferably 2 to 20 walls.

The diameter of the carbon nanotube is preferably 0.3 to 200 nm, more preferably 1 to 100 nm, and further preferably 2 to 50 nm. The cross section of the carbon nanotube may be substantially circular, elliptical, n-gonal (n is an integer of 3 or more), and the like.

The number of walls and the number distribution of the walls of the carbon nanotubes described above can be measured based on, for example, a captured image obtained by a scanning electron microscope (SEM) or a transmission electron microscope (TEM).

Next, a method of producing a carbon nanotube assembly according to one embodiment of the present invention will be described. The carbon nanotube assembly can be produced, for example, by forming a catalyst layer on a substrate, supplying a carbon source with the catalyst activated by heat, plasma, etc., and growing carbon nanotubes on the substrate. Then, the carbon nanotube assembly can be preferably produced by Chemical Vapor Deposition (CVD) Method. This method is capable of producing a carbon nanotube assembly that is oriented substantially vertically to the substrate, that is, the length of each carbon nanotube extends from one surface to the other surface.

Any appropriate thermal CVD apparatus can be adopted as an apparatus for producing the carbon nanotube assembly. For example, as shown in FIG. 2, a hot wall-type thermal CVD apparatus 30 constituted by surrounding a cylindrical reaction vessel 31 with a resistance heating type electric tubular furnace 32 may be used. In that case, as the reaction vessel 31, for example, a heat-resistant quartz tube or the like is preferably used. A substrate (a substrate for growing) S can be arranged in such an apparatus 30 and the carbon nanotube assembly 100 can be grown thereon.

As the substrate S (FIG. 2) used in the method for producing the carbon nanotube assembly 100, for example, a material that has a smooth surface and that is able to withstand the high temperatures of the carbon nanotube production process may be used. Examples of such materials include metal oxides such as quartz glass, zirconia and alumina; metals such as silicon (silicon wafer etc.), aluminum, and copper; carbides such as silicon carbide; nitrides such as silicon nitride, aluminum nitride, and gallium nitride.

In the production of a carbon nanotube assembly, as described above, a catalyst layer is formed on a substrate, and examples of the material of the catalyst layer include metal catalysts such as iron, cobalt, nickel, gold, platinum, silver, copper, and the like.

When a carbon nanotube assembly is produced, an intermediate layer may be provided between the substrate and the catalyst layer, as needed. Examples of the materials constituting the intermediate layer include metals and metal oxides. In one embodiment, the intermediate layer is constituted by an alumina/hydrophilic film.

As a method of forming the alumina/hydrophilic film, for example, a SiO₂ film (hydrophilic film) is first formed on a substrate. Then, Al is vapor-deposited and oxide the SiO₂ by heating the film, for example, up to 450° C. to form Al₂O₃. According to this production method, Al₂O₃ interacts with the hydrophilic SiO₂ film to form a film with Al₂O₃ surface having different particle size from that of a film obtained by directly depositing Al₂O₃. The film with Al₂O₃ surface having different particle diameter is easily to be formed by forming a hydrophilic film on the substrate. Further, the film with Al₂O₃ surface having different particle diameter is easily to be formed by depositing Al followed by oxidizing to Al₂O₃, compared with the case where Al₂O₃ is deposited directly.

The amount of the catalyst layer that can be used for producing the carbon nanotube assembly is preferably 50 to 3000 ng/cm², more preferably 100 to 2000 ng/cm², and particularly preferably 200 to 2000 ng/cm². The carbon nanotube assembly having the low orientation portion can be easily formed by adjusting the amount of the catalyst layer within the above range for producing the carbon nanotube assembly.

Any appropriate method can be adopted as a method for forming the catalyst layer. For example, a method of depositing a metal catalyst by EB (electron beam), sputtering, and the like, a method of applying a suspension of metal fine particle catalysts on a substrate can be mentioned.

The catalyst layer formed by the above method can be atomized by heat treatment or the like. For example, the temperature of the heat treatment is preferably 400 to 1200° C., more preferably 500 to 1100° C., further preferably 600 to 1000° C., and particularly preferably 700 to 900° C. For example, the catalyst layer is subjected to the heat treatment for more than 0 minutes to 180 minutes, more preferably 5 to 150 minutes, further preferably 10 to 120 minutes, and particularly preferably 15 to 90 minutes. In one embodiment of the present invention, a carbon nanotube assembly in which a low orientation portion in the carbon nanotube assembly is appropriately formed by the above-mentioned heat treatment can be obtained. For example, the average particle diameter of the circle-equivalent diameter of the fine particle catalysts formed by the method such as the above heat treatment may be 1 μm or less. This average particle diameter is preferably 1 to 300 nm, more preferably 3 to 100 nm, further preferably 5 to 50 nm, and particularly preferably 10 to 30 nm. In one embodiment of the present invention, the carbon nanotube assembly, in which a low orientation portion is formed, can be easily obtained as long as the size of the fine particle catalysts is in the above range.

Examples of the carbon source that can be used for producing the carbon nanotube assembly include hydrocarbons such as methane, ethylene, acetylene, benzene, and the like; alcohols such as methanol, ethanol, and the like. The formation of the low orientation portion in the carbon nanotube assembly can be controlled depending on the type of carbon source used. For example, a low orientation portion having a structure (mesh structure) suitable as an electrode material for a sensor is easily formed by using ethylene as the carbon source. The carbon source can be supplied as a mixed gas with one or more of helium, hydrogen, and water vapor. In one embodiment of the present invention, the formation of low orientation portion can be controlled by a composition of mixed gas. For example, a low orientation portion having a more apparent mesh structure can be formed by increasing the amount of hydrogen in the mixed gas.

The concentration of the carbon source (preferably ethylene) in the mixed gas at 23° C. is preferably 2 to 30 vol % (volume %) and more preferably 2 to 20 vol %. The concentration of helium in the mixed gas at 23° C. is preferably 15 to 92 vol % and more preferably 30 to 80 vol %. The concentration of hydrogen at 23° C. in the mixed gas is preferably 5 to 90 vol % and more preferably 20 to 90 vol %. The concentration of water vapor at 23° C. in the mixed gas is preferably 0.02 to 0.3 vol % and more preferably 0.02 to 0.15 vol %. A low orientation portion can be formed in which the portion includes a suitable structure as an electrode material for a sensor by using the mixed gas having the above composition.

The volume ratio of the carbon source (preferably ethylene) and the hydrogen in the mixed gas at 23° C. (volume of hydrogen/volume of carbon source) is preferably 2 to 20 and more preferably 4 to 10. In the mixed gas, the volume ratio of water vapor and hydrogen at 23° C. (volume of hydrogen/volume of water vapor) is preferably 100 to 2000 and more preferably 200 to 1500. Within the above range, a carbon nanotube assembly having a low orientation portion having a structure suitable as a sensor electrode material can be formed.

The carbon nanotube assembly is preferably produced at a temperature of 400 to 1000° C., more preferably 500 to 900° C., further more preferably 600 to 800° C., and most preferably 700 to 800° C. A formation of low orientation portion can be controlled by the production temperature. At least either one of atomizing the catalyst or growing the carbon nanotubes can be performed at the above temperature. The temperatures of atomizing the catalyst and growing the carbon nanotubes may be the same or different, but are preferably the same.

In one embodiment of the present invention, as described above, the catalyst layer is formed on the substrate, the carbon source is supplied in the state where the catalyst is activated. After the carbon nanotubes are grown on the substrate, the supply of the carbon source is stopped and the carbon nanotubes are maintained at the reaction temperature in the presence of a carbon source. The formation of the low orientation portion can be controlled by the conditions of this reaction temperature maintaining step.

A catalyst layer is formed on the substrate, a carbon source is supplied in a state where the catalyst is activated. After the carbon nanotubes are grown, a predetermined load in the thickness direction of the carbon nanotubes on the substrate may be applied so as to compress the carbon nanotubes. By doing so, a carbon nanotube assembly which is constituted by only a low orientation portion of carbon nanotubes or a large proportion of low orientation portion, or the carbon nanotube assembly is constituted by a low orientation portion. The load is, for example, 1 to 10000 g/cm², preferably 5 to 1000 g/cm², and more preferably 100 to 500 g/cm². The thickness of the carbon nanotube walls after compression (that is, the carbon nanotube assembly) is 10 to 90% and preferably 20 to 80% with respect to the thickness of the carbon nanotube walls before compression.

The carbon nanotube assembly is obtained by forming (growing) on the substrate as described above, and then the carbon nanotube assembly is separated from the substrate. As described above, the carbon nanotube assembly according to the present embodiment has the low orientation portion, and thus the carbon nanotube assembly can be obtained in the sheet-like form on the substrate.

As described above, the carbon nanotube assembly may be obtained by growing a plurality of carbon nanotubes on fine particle catalysts in which the fine particle catalysts have an average particle diameter of 1 μm or less that are arranged on a planar substrate, and the grown plurality of carbon nanotubes are separated from the substrate.

The electrode material equipped with the carbon nanotube assembly according to the present embodiment can be used, for example, by adhering to a substrate for electrode. That is, the present embodiment may be an electrode for a sensor in which the above-mentioned electrode for a sensor is adhered to the electrode substrate.

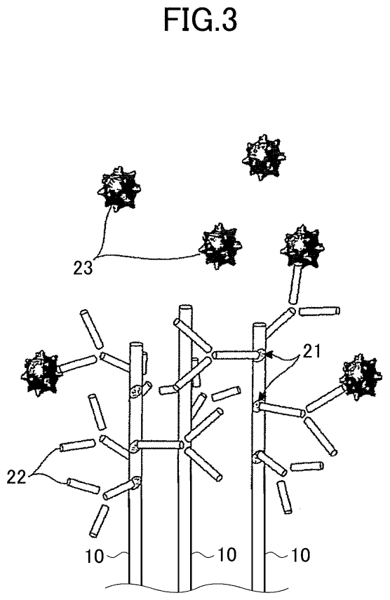

The electrode material for a sensor according to the present embodiment can be suitably used in a biosensor, particularly in a sensor that detects a bio-related substance such as an antigen, an amino acid, a protein, a nucleic acid, an enzyme as a target to be detected. In this case, at least one kind of substance on at electrode side such as an antibody, protein, sugar, enzyme, nucleic acid or the like that can biologically react with the target to be detected can be immobilized on a portion of the surface of the electrode material. In the immobilization, for example, as shown in FIG. 3, a linker 21 may be attached to the tip of each carbon nanotube 10, and the substance at the electrode side 22 may be immobilized on the linker 21. With such a configuration, the target to be detected 23, which biologically reacts with the substance at the electrode side 22, can be detected.

Further, the electrode material for a sensor according to the present embodiment can also be used in a chemical sensor for detecting various ions and molecules other than the above-mentioned bio-related substances, particularly VOC, carbonized oxygen, oxygen nitride, and the like.

Thus, one embodiment of the present invention may be a sensor provided with the above-mentioned electrode material for a sensor, or a biosensor provided with the above-mentioned electrode material for a sensor.

### EXAMPLES

Hereinafter, the present invention will be described based on examples, but the present invention is not limited thereto.

### [1. Evaluation of Structure of Carbon Nanotube Assembly]

### <Production of Carbon Nanotube Assembly>

### Example 1

An Al₂O₃ thin film of 4000 ng/cm² was formed on a silicon substrate (manufactured by VALQUA FT Inc., thickness 700 μm) by a sputtering device (manufactured by Shibaura Mechatronics Corp., trade name “CFS-4ES”) (ultimate vacuum: 8.0×10⁻⁴ Pa, sputtering gas: Ar, gas pressure: 0.50 Pa). On this Al₂O₃ thin film, an Fe thin film of 260 ng/cm² was further supported as a catalyst layer by the above sputtering apparatus (sputtering gas: Ar, gas pressure: 0.75 Pa).

The substrate on which the catalyst layer was formed was mounted in a 30 mmφ quartz tube, and a helium/hydrogen (105/80 sccm) mixed gas kept at a moisture content of 700 ppm was flowed in the quartz tube for 120 minutes. At that time, the temperature in the tube was adjusted to 865° C. by using an electric tubular furnace, and Fe in the catalyst layer was atomized. The density of the Fe particles was 500 particles/pmt.

Furthermore, after the temperature was lowered to 765° C., the gas in the tube such as a mixed gas of helium/hydrogen/ethylene/water (volume ratio of 34.4/65/0.5/0.1) was flowed at 1800 sccm for 60 minutes so as to grow the carbon nanotubes on the substrate. The raw material gas was stopped, and the mixed gas of helium/hydrogen (105/80 sccm) having a water content of 1000 ppm was cooled to room temperature while flowing in the quartz tube.

By the above operation, a sheet-like carbon nanotube assembly having a thickness of 500 μm was obtained. The resulting carbon nanotube assembly was separated from the silicon substrate by using tweezers.

### Example 2

A carbon nanotube assembly was obtained in the same manner as Example 1 except that the time for atomizing Fe in the catalyst layer was changed to 30 minutes. The thickness of the obtained carbon nanotube assembly was 700 μm. The density of Fe fine particles after the atomization of Fe was 567 particles/μm².

### Example 3

A carbon nanotube assembly was obtained in the same manner as Example 1 except that the amount of the supported catalyst was changed to 550 ng/cm² and the time for atomizing Fe in the catalyst layer was changed to 30 minutes. The thickness of the obtained carbon nanotube assembly was 700 μm. The density of Fe fine particles after the atomization of Fe was 583 particles/μm².

### Example 4

A carbon nanotube assembly was obtained in the same manner as Example 1 except that the amount of the supported catalyst was changed to 550 ng/cm², the temperature for atomizing Fe in the catalyst layer was changed to 765° C., the time for atomizing Fe in the catalyst layer was changed to 30 minutes, and the gas used for growing the carbon nanotubes was changed to a mixed gas of helium/hydrogen/ethylene/water (69.9/22/8/0.1 in volume ratio). The thickness of the obtained carbon nanotube assembly was 1000 μm. The density of Fe fine particles after the atomization of Fe was 917 particles/μm².

### Example 5

A carbon nanotube assembly was obtained in the same manner as Example 1 except that the amount of the supported catalyst was changed to 550 ng/cm², the atomization of Fe of the catalyst layer was not carried out, and the gas used for growing the carbon nanotubes was changed to a mixed gas of helium/hydrogen/ethylene/water (48.92/43/8/0.08 in volume ratio). The thickness of the obtained carbon nanotube assembly was 1000 μm. The density of Fe fine particles after the atomization of Fe was 1050 particles/μm².

### Example 6

A carbon nanotube assembly was obtained in the same manner as Example 5 except that the gas used for growing the carbon nanotubes was changed to a mixed gas of helium/hydrogen/ethylene/water (48.97/43/8/0.03 in volume ratio). The thickness of the obtained carbon nanotube assembly was 1000 μm. The density of Fe fine particles after the atomization of Fe was 1050 particles/μm².

### Example 7

A carbon nanotube assembly was obtained in the same manner as Example 5 except that the gas used for growing the carbon nanotubes was changed to a mixed gas of helium/hydrogen/ethylene/water (59.9/32/8/0.1 in volume ratio). The thickness of the obtained carbon nanotube assembly was 1000 μm. The density of Fe fine particles after the atomization of Fe was 1050 particles/μm².

### Example 8

A carbon nanotube assembly was obtained in the same manner as Example 5 except that the gas used for growing the carbon nanotubes was changed to a mixed gas of helium/hydrogen/ethylene/water (15.9/65/19/0.1 in volume ratio). The thickness of the obtained carbon nanotube assembly was 1000 μm. The thickness of the obtained carbon nanotube assembly was 1200 μm. The density of Fe fine particles after the atomization of Fe was 1050 particles/μm².

### Example 9

A carbon nanotube assembly was obtained in the same manner as Example 1 except that the amount of the supported Fe catalyst was changed to 1100 ng/cm², the time for atomizing Fe in the catalyst layer was changed to 30 minutes, and the gas used for growing the carbon nanotubes was changed to a mixed gas of helium/hydrogen/ethylene/water (26.9/65/0.1/8 in volume ratio). The thickness of the obtained carbon nanotube assembly was 700 μm. The density of Fe fine particles after the atomization of Fe was 608 particles/μm².

### Example 10

A carbon nanotube assembly was obtained in the same manner as Example 9 in the same manner as Example 1 except that the amount of the supported Fe catalyst was changed to 1650 ng/cm². The thickness of the obtained carbon nanotube assembly was 700 μm. The density of Fe fine particles after the atomization of Fe was 608 particles/μm².

### Comparative Example 1

A multi-wall carbon nanotube array (model number: “NTA05”) manufactured by Hamamatsu Carbonics Corporation was prepared as the carbon nanotube assembly of Comparative Example 1. The carbon nanotube assembly is provided in the sheet-like form in which carbon nanotubes are stretched in a substantially vertical direction.

The orientation degree of carbon nanotubes in the carbon nanotube assembly of Examples 1 to 9 and Comparative Example 1 were measured in the vicinity of the main surface and in the intermediate portion in the thickness direction of the carbon nanotube assembly. The measurement method of the orientation degree is the following.

### <Measurement of Orientation Degree>

First, a cross-section of carbon nanotube assembly cut perpendicular to the plane direction was imaged by using a scanning electron microscope (SEM), and a cross-sectional image of the area of 4 μm length×6 μm width at 20,000 times magnification was obtained. In the obtained cross-sectional image, a carbon nanotube was regarded as a needle shape particle by using a device for measuring a needle shape particle measurement of WinROOF2015 (manufactured by Mitani Corporation) so as to calculate the length and the orientation degree of a needle shape particles. According to this measurement function, overlapping needle shape particle can be individually separated and the individual needle particle can then be measured. The calculation was performed according to the following procedure.

1. Background removal: Object size 0.248 μm
2. Processed by median filter: Filter size 3*3
3. Look-up table conversion (histogram average brightness correction), Correction reference value: 90
4. Binarization with a single threshold: Threshold 90, transparency 53
5. Number of morphological processing (closing processing): 1
6. Needle shape particle separation measurement, Minimum measurement length: 0.49630 μm, Maximum measurement width: 0.4963 μm

Next, one needle shape particle was divided each time the calculated orientation angle changed by 10°, and the length of each divided portion of the needle shape particle was obtained. Then, in each of the divided portion of the needle shape particle, the percentage of the total length of the portions having an orientation angle of 70° to 110° with respect to 100% of the length of the entire needle shape particle (the total length of the needle shape particle portion of orientation angle in the range of 70° to 110°/the total length of the entire needle shape particle) was determined. The above-mentioned orientation angle is an angle with respect to the main surface (lower surface or upper surface) of the sheet-like carbon nanotube assembly.

When measuring the orientation degree in the vicinity of the main surface, an image in which the position of 2 μm from the main surface on the side where the catalyst layer was formed (the side of silicon substrate) in the production process to be positioned as a center of the image was obtained (i.e., an image of up to 4 μm from the surface of the main surface was obtained). Further, when measuring the orientation degree of the intermediate portion, an image in which the center position in the thickness direction as a center of the image was obtained. The results of the measured orientation degree are shown in Table 1.

<Measuring the outer diameter of carbon nanotubes>

A cross-section of carbon nanotube assembly cut perpendicular to the plane direction was imaged by using a scanning electron microscope (SEM), and 20,000 times enlarged cross-sectional image was obtained. The outer diameter of each carbon nanotube in the image was measured and the average was obtained. The images were obtained in the vicinity of 2 μm from the main surface facing the silicon substrate and in the vicinity of 2 μm from the main surface facing away from the silicon substrate. Then, the average of both was calculated.

### <Measurement of the Number of Walls of Carbon Nanotubes>

The number of walls was confirmed based on the SEM image in the same manner as the above-described measurement of the outer diameter. In the same manner as the measurement of the outer diameter, the number of walls was determined in the vicinity of 2 μm from the main surface facing the silicon substrate and in the vicinity of 2 μm from the main surface facing away from the silicon substrate. Then, the average of both was calculated.

TABLE 1

| | | | | | | | | | | | | Comparative |
|---|---|---|---|---|---|---|---|---|---|---|---|---|
| | | Example | | | | | | | | | | Example |
| | | 1 | 2 | 3 | 4 | 5 | 6 | 7 | 8 | 9 | 10 | 1 |
| Condition of | Supporting amount of | 260 | 260 | 550 | 550 | 550 | 550 | 550 | 550 | 1100 | 1650 | — |
| atomization | Fe catalyst (ng/cm²) | | | | | | | | | | | |
| of Fe | Temperature (° C.) | 865 | 865 | 865 | 765 | — | — | — | — | 865 | 865 | — |
| | Time (min) | 120 | 30 | 30 | 30 | — | — | — | — | 30 | 30 | — |
| | Density of Fe fine | 500 | 567 | 583 | 917 | 1050 | 1050 | 1050 | 1050 | 608 | 608 | — |
| | particle (number of | | | | | | | | | | | |
| | particles/μm²) | | | | | | | | | | | |
| Component of | H₂ | 65% | 65% | 65% | 22% | 43% | 43% | 32% | 65% | 65% | 65% | — |
| gas used for | C₂H₄ | 8% | 8% | 8% | 8% | 8% | 8% | 8% | 19% | 8% | 8% | — |
| growing (in | H₂O | 0.10% | 0.10% | 0.10% | 0.10% | 0.08% | 0.03% | 0.10% | 0.10% | 0.10% | 0.10% | — |
| terms of vol %) | | | | | | | | | | | | |
| Thickness (μm) | | 500 | 700 | 700 | 1000 | 1000 | 1000 | 1000 | 1200 | 700 | 700 | 500 |
| Orientation | Intermediate portion | 40.0 | 73.5 | 79.8 | 79.4 | 84.4 | 84.0 | 86.5 | 88.0 | 82.1 | 84.3 | 84.6 |
| degree | Vicinity of the main | 30.0 | 32.0 | 46.9 | 56.9 | 41.9 | 61.9 | 38.8 | 58.3 | 48.8 | 50.2 | 77.4 |
| | surface | | | | | | | | | | | |

| | | | | | | | | | | | |
|---|---|---|---|---|---|---|---|---|---|---|---|
| Average of outer diameter of CNT (nm) | 4.8 | 5.0 | 5.1 | 7.6 | 7.3 | 7.2 | 7.4 | 7.1 | 13.4 | 13.1 | 32.0 |
| Average of number of walls of CNT | 10.6 | 11.8 | 11.8 | 3.1 | 2.9 | 3.0 | 3.0 | 2.9 | 6.9 | 9.1 | 17.0 |

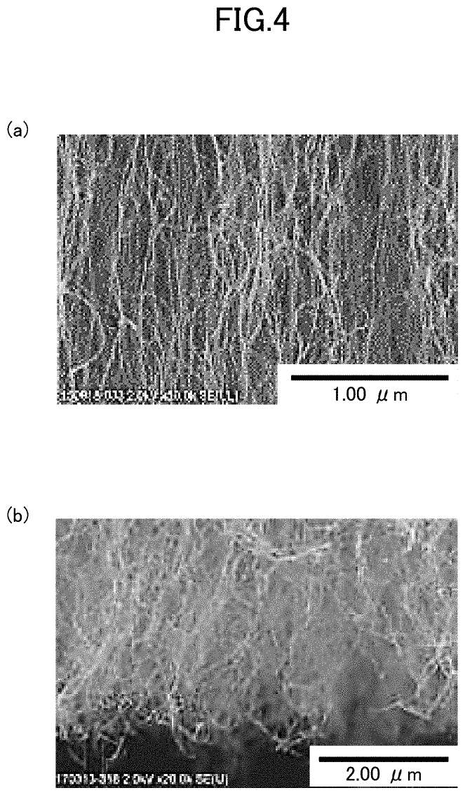

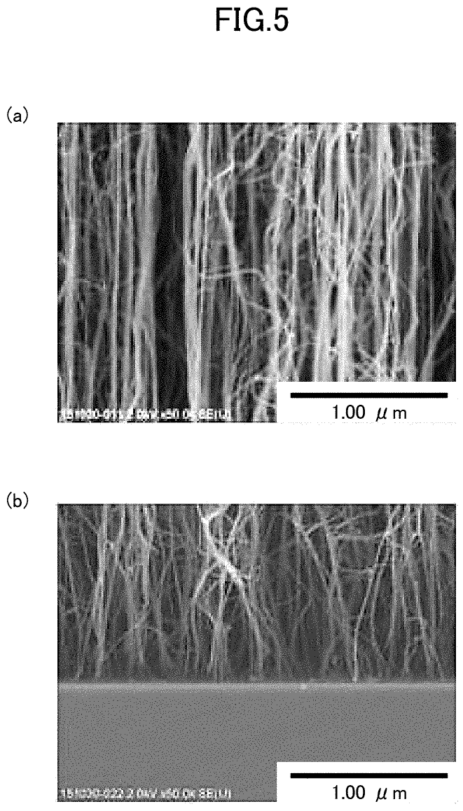

Further, FIG. 4(a) shows a cross-sectional image of the intermediate portion and FIG. 4(b) shows a cross-sectional image in the vicinity of the main surface in Example 3. FIG. 5(a) shows a cross-sectional image of the intermediate portion and FIG. 5(b) shows a cross-sectional image in the vicinity of the main surface in Comparative Example 1. In the carbon nanotube assembly of the present embodiment, a plurality of carbon nanotubes is entangled with each other to form a mesh structure in the vicinity of the main surface of the sheet.

### [2-1. Evaluation of Detection Sensitivity of Sensor]

### <Production of Electrode>

### Example 11

The evaluation was performed using a round-shaped carbon electrode manufactured by BioDevice Technology Limited. The carbon electrode was a three-electrode printed electrode (working electrode: carbon, reference electrode: Ag/AgCl, working electrode area: 2.64 mm²). The sheet-like carbon nanotube assembly of Example 4 was attached to the working electrode of the round-shaped carbon electrode with a carbon paste (“G7711” manufactured by EM Japan Co., Ltd.). The solvent of the carbon paste was dried to obtain an electrode.

### Comparative Example 2

The electrode, which the sheet-like carbon nanotube assembly was not attached in Example 11, was used as Comparative Example 2.

### <Cv Measurement>

In each electrode of Example 11 and Comparative Example 2, 40 μL of an electrolytic solution PBS (phosphate buffer solution) containing potassium ferricyanide at 1 mM was dropped so that the counter electrode, working electrode, and reference electrode were covered with the solution. Then, CV (cyclic voltammogram) was measured at a sweep rate of 0.1 V/s. At that time, an electron transfer (oxidation-reduction pair: [Fe(CN)⁶]³⁻/[Fe(CN)⁶]⁴⁻) during the oxidation/reduction of potassium ferricyanide can be detected as an electric current value. The electric current value (μA) of the reduction peak was determined from the obtained CV. Further, the electric current value per area of the working electrode was obtained, and the electric current value per area was also obtained when that of Comparative Example 2 was set to 1. The results are shown in Table 2.

TABLE 2

| | | Electric current | Electric current | |
|---|---|---|---|---|
| | Area of working | value at reduction | value per area | |
| | electrode (mm²) | peak (μA) | (μA/mm²) | Comparison*¹ |
| Comparative Example 2 | 2.64 | 2.1 | 0.795 | 1 |
| Example 11 | 2.64 | 16.9 | 6.386 | 8.03 |

*¹Comparaive value with Comparative Example 2 as “1”

From Table 2, Example 11 in which the carbon nanotube assembly was used as an electrode according to one embodiment of the present invention showed that the detection sensitivity was improved by about 10 times as compared with Comparative Example 2 in which a carbon nanotube assembly was not used.

### [2-2. Evaluation of Detection Sensitivity of Sensor]

### <Production of Electrode>

### Example 12

A pyrene derivative (1-pyrenebutyric acid N-hydroxysuccinimide ester) as a linker was dissolved in DMF (N,N-dimethylformamide) and adjusted to 1 mM. This solution was dropped on the carbon nanotube assembly of Example 9 and left at room temperature for 1 hour. Then, the carbon nanotube assembly was washed with an acetone solvent and dried to prepare a pyrene derivative-coated carbon nanotube sheet. The obtained sheet was attached onto a working electrode of a round-shaped carbon electrode manufactured by BioDevice Technology Inc. (electrode printing of three electrode system, working electrode: carbon, reference electrode: Ag/AgCl, area of working electrode: 2.64 mm²) by a Carbon paste (“G7711” manufactured by EM Japan Co., Ltd.). The solvent of the carbon paste was dried to obtain a pyrene derivative-coated electrode.

A solution of the Au nanoparticle-antibody complex was dropped on the obtained electrode. Then, the electrode was left stand for 20 minutes and then washed with water. According to this treatment, the ester portion of pyrene was replaced with the antibody with Au marker.

### Comparative Example 3

A pyrene derivative-coated electrode was obtained in the same manner as Example 13 except that the prepared DMF solution of the pyrene derivative used in Example 12 was dropped directly onto the round-shaped carbon electrode. Then, the ester portion of pyrene was replaced with the antibody with Au marker in the same manner as in Example 13

### <Cv Measurement>

CV of the electrode with an antibody having an Au marker obtained in Example 12 and Comparative Example 3 were measured in 0.1 M hydrochloric acid at a sweep rate of 0.1 V/s. At the time of the measurement, the electric current value due to Au was confirmed for each of Example 13 and Comparative Example 3. Specifically, the CV at the electrode of Example 13 in which the solution of the Au nanoparticle-antibody complex was not dropped to the electrode and the CV at the electrode in Example 13 were measured, respectively. It was confirmed that there was no peak around 0.2 to 0.3V in the former measurement. The baseline was drawn with the waveform of the current peak value in Example 13, and the difference from the peak value was used as the detected current. In Comparative Example 3, the detected current was similarly obtained. The results are shown in Table 3. All the detected current in Table 3 are the electric current value in the first cycle.

TABLE 3

| | Detected current (μA) |
|---|---|
| Comparative Example 3 | 0.89 |
| Example 12 | 57 |

According to Table 3, the detected current of Example 12 was found to be 60 times or more compared to that of Comparative Example 3.

### [2-3. Evaluation of Detection Sensitivity of a Sensor]

### <Production of Electrode>

### Example 13

The surface of the Si wafer was covered with Au by a sputtering to obtain a conductive substrate of 2 cm×2 cm. Then, the sheet-like carbon nanotube assembly produced in Example 3 was attached onto the surface covered by Au with a carbon paste (“G7711” manufactured by EM Japan Co., Ltd.) to obtain an electrode.

### Comparative Example 4

In Example 13, an electrode to which a sheet-like carbon nanotube assembly was not attached, that is, an electrode in which the surface of the Si wafer coated with Au by a sputtering was prepared.

### Comparative Example 5

The electrode coated with glassy carbon was prepared on the conductive substrate used in Example 13.

### <CV Measurement>

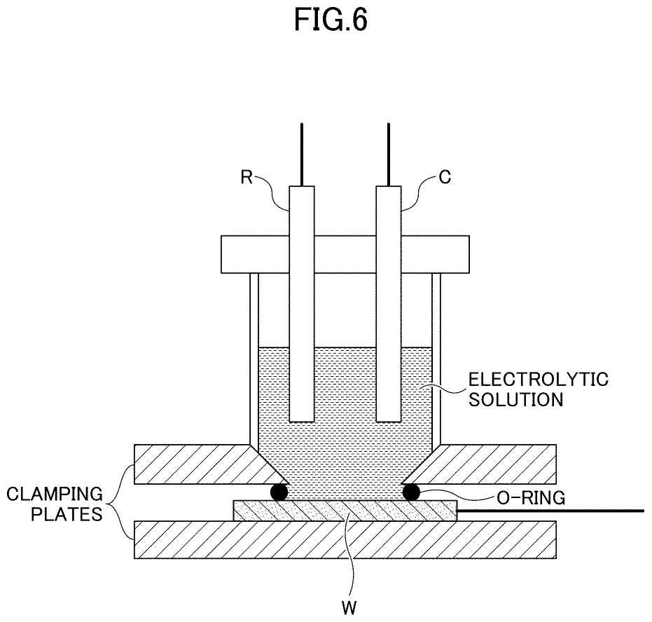

CV was measured by a sensor 50 in which the sensor 50 was produced by each electrode obtained in Example 13, Comparative Example 4, and Comparative Example 5 as working electrodes W, Pt as a counter electrode C, and Ag/AgCl as a reference electrode R as shown in FIG. 6. In the configuration as shown in FIG. 6, the exposed area of the working electrode to the electrolytic solution is defined by the O-ring (diameter of 5 mm, area of 0.2 cm²). The working electrode W, the counter electrode C, and the reference electrode R were immersed in an electrolyte solution (2 mM potassium ferricyanide, 200 mM sodium sulfate), and CV measurement was carried out at a sweep rate of 0.1 V/s. Table 4 shows the current value of the reduction peaks of Example 13, Comparative Examples 4, and Comparative Examples 5.

TABLE 4

| | Electric current value |
|---|---|
| | at reduction peak |
| | (μA/cm²) |
| Example 13 | 3600 |
| Comparative Example 4 | 121 |
| Comparative Example 5 | 139 |

According to Table 4, it was found that the current value of the reduction peak by the electrode of Example 13 was 30 times or more of the electrodes of Comparative Examples 4 and 5.

This application is based on and claims priority of Japanese Patent Application No. 2018-020761 filed Feb. 8, 2018, the entire contents of which are hereby incorporated by reference.

### DESCRIPTION OF THE REFERENCE NUMERALS

10: Carbon nanotubes
11: One surface
12: The other surface
21: Linker
22: Bio-related substance
23: Target to be detected
30: Production apparatus of carbon nanotube assembly
31: Reaction vessel
32: Electric tubular furnace
110: Low orientation portion
120: High orientation portion
100: Carbon nanotube assembly
C: Counter electrode
R: Reference electrode
S: Substrate for growing
W: Working electrode

1. An electrode material for a sensor, the material comprising:
a sheet-like carbon nanotube assembly including a plurality of carbon nanotubes,
wherein a length of each carbon nanotube extends from one surface of the carbon nanotube assembly toward the other surface thereof, and the carbon nanotube assembly includes a low orientation portion of the carbon nanotubes.

2. The electrode material for a sensor according to claim 1,
Wherein the low orientation portion include a distribution of orientation angles of the carbon nanotubes, and an orientation degree of the carbon nanotubes is 75% or less, and
the orientation degree is a proportion of the total length of the portions of the carbon nanotubes having orientation angle of 70 to 110° with respect to the one surface or the other surface based on 100% of the total length of the carbon nanotubes.

3. The electrode material for a sensor according to claim 1,
wherein the low orientation portion is formed at an area up to 20 μm from the one surface of the carbon nanotube assembly in the thickness direction.

4. The electrode material for a sensor according to claim 1,
wherein the one surface is attached to an electrode substrate in a sensor.

5. The electrode material for a sensor according to claim 1,
wherein the sheet has a thickness of 10 to 2000 μm.

6. The electrode material for a sensor according to claim 1,
wherein the carbon nanotubes are multi-walled carbon nanotubes.

7. The electrode material for a sensor according to claim 1,
wherein the carbon nanotube assembly is obtained by growing a plurality of carbon nanotubes on fine particle catalysts in which the fine particle catalysts have an average particle diameter of 1 μm or less that are arranged on a planar substrate, and the grown plurality of carbon nanotubes are separated from the substrate.

8. An electrode for a sensor, wherein the electrode material for a sensor of claim 1 is adhered to a substrate for an electrode.

9. A sensor comprising the electrode material for a sensor of claim 1.

10. A biosensor comprising the electrode material for a sensor of claim 1.
